(19) United States
(12) Patent Application Publication
(10) Pub. No.: US 2025/0177420 A1
(43) Pub. Date: Jun. 5, 2025

(54) BCL-2 INHIBITORS AND AURORA KINASE INHIBITORS FOR TREATING CANCER

(71) Applicants: Genome Research Limited, London (GB); Stichting Het Nederlands Kanker Instituut-Antoni Van Leeuwenhoek Ziekenhuis, Amsterdam (NL)

(72) Inventors: Elizabeth Coker, London (GB); Amy Emery, Cambridge (GB); Mathew Garnett, London (GB); Patricia Jaaks, London (GB); Daniel Vis, Amsterdam (NL); Lodewyk Wessels, Amsterdam (NL)

(73) Assignees: Genome Research Limited, London (GB); Stichting Het Nederlands Kanker Instituut-Antoni Van Leeuwenhoek Ziekenhuis, Amsterdam (NL)

(21) Appl. No.: 18/836,999

(22) PCT Filed: Feb. 10, 2023
(86) PCT No.: PCT/EP2023/053392

§ 371 Date: Aug. 8, 2024

(30) Foreign Application Priority Data
Feb. 11, 2022 (GB) 2201824.6

(51) Int. Cl. A61K 31/635; A61K 31/437; A61K 31/4545; A61K 31/506; A61K 31/5377; A61K 31/55; A61K 31/675; A61P 35/00

(52) U.S. Cl. CPC A61K 31/635; A61K 31/437; A61K 31/4545; A61K 31/506; A61K 31/5377; A61K 31/55; A61K 31/675; A61P 35/00

(57) ABSTRACT

The present invention relates to a combination therapy of an inhibitor of the Bel-2 family of proteins selected from navitoclax, venetoclax, A-1331852, AZD5991, or A-1155463, together with an aurora kinase inhibitor for use in a method of treatment of a cancer selected from breast, ovarian, pancreatic, or prostate cancer in a patient.

### FIELD OF THE INVENTION

The invention relates to combination therapies for the treatment of cancer.

### BACKGROUND

Despite advances in treatment cancer continues to have a major impact on societies, families, and individuals across the world. Cancer is among the leading causes of death worldwide. According to statistics provided by the National Cancer Institute, in 2018 there were 18.1 million new cases and 9.5 million cancer-related deaths worldwide. By 2040 the number of new cancer cases per year is expected to rise to 29.5 million and the number of cancer-related deaths to 16.4 million.

The use of single-agent targeted therapies in patients with molecularly-defined tumours is improving cancer treatment. Nonetheless, many patients still lack effective treatments and pre-existing or acquired resistance limits the clinical benefit of even our most advanced medicines. Combination therapies using the growing number of targeted anti-cancer agents have potential to overcome resistance, to enhance response to existing drugs, to reduce dose limiting single agent toxicity, and to expand the range of treatments for patients.

### SUMMARY OF THE INVENTION

The invention is directed to the use of therapeutic combinations of active ingredients for the treatment of cancer in patients, and especially to the combination of an inhibitor of the Bcl-2 family of proteins together with an aurora kinase inhibitor. As described herein, the inventors have observed synergy for combinations of inhibitors of the Bcl-2 family of proteins, such as navitoclax, with aurora kinase inhibitors in cancer cell lines.

In a first aspect, the invention may provide a combination of an inhibitor of the Bcl-2 family of proteins selected from navitoclax, venetoclax, A-1331852, AZD5991, or A-1155463, together with an aurora kinase inhibitor for use in a method of treatment of a cancer selected from breast, ovarian, pancreatic, or prostate cancer in a patient.

In some cases, the invention may provide an inhibitor of the Bcl-2 family of proteins selected from navitoclax, venetoclax, A-1331852, AZD5991, or A-1155463, for use in a method of treatment of a cancer selected from breast, ovarian, pancreatic, or prostate cancer in a patient, and wherein the inhibitor of the Bcl-2 family of proteins is administered to the patient in combination with an aurora kinase inhibitor.

In some cases, the invention may provide an aurora kinase inhibitor for use in a method of treatment of a cancer selected from breast, ovarian, pancreatic, or prostate cancer in a patient, and wherein the aurora kinase inhibitor is administered to the patient in combination with an inhibitor of the Bcl-2 family of proteins selected from navitoclax, venetoclax, A-1331852, AZD5991, or A-1155463.

In some embodiments, navitoclax is used. In some embodiments, venetoclax is used. In some embodiments, A-1331852 is used. In some embodiments, AZD5991 is used. In some embodiments, A-1155463 is used.

In some embodiments, the cancer is non-HER2-enriched breast cancer. The inventors have observed significant synergy across non-HER2 breast cancer cell lines. In some embodiments, the cancer is determined to be HER2−.

In some embodiments, the cancer is basal-like breast cancer. In some embodiments, the cancer is triple negative breast cancer.

In some embodiments, the cancer is luminal A breast cancer.

In some embodiments, the cancer is luminal B breast cancer.

In some embodiments, the aurora kinase inhibitor is an aurora A kinase inhibitor. In some embodiments, the aurora kinase inhibitor is selected from alisertib, tozasertib, ZM447439, AZD2811 (also known as barasertib-hQPA), AZD1152 (barasertib, a prodrug of barasertib-hQPA), LY3295668, MK-5108, GSK1070916, and MLN8054. In some embodiments, the aurora kinase inhibitor is selected from alisertib, tozasertib, ZM447439, AZD2811, and AZD1152. In some embodiments, the aurora kinase inhibitor is selected from alisertib, tozasertib, and ZM447439. In some embodiments, the aurora kinase inhibitor is alisertib.

It will be understood that the inhibitor of the Bcl-2 family of proteins selected from navitoclax, venetoclax, A-1331852, AZD5991, or A-1155463 and the aurora kinase inhibitor may be administered together or separately and may be administered at the same time or at different times. For example, the compounds may be administered on different days as part of a treatment cycle or treatment regimen. Suitably but not necessarily the inhibitor of the Bcl-2 family of proteins described herein and the aurora kinase inhibitor will be formulated separately. In preferred methods, both compounds are formulated for oral administration.

The combination therapies claimed may be used both curatively and palliatively. They may lead to better patient outcomes and/or experiences when compared to other treatment regimens and additionally or alternatively may expand the treatment options available to patients.

Suitably, the patient may be a human patient.

The invention also relates to a method of treatment of cancer in a patient in need thereof, wherein the method comprises the step of administering a combination of an effective amount of an inhibitor of the Bcl-2 family of proteins selected from navitoclax, venetoclax, A-1331852, AZD5991, or A-1155463, together with an effective amount of an aurora kinase inhibitor to the patient, wherein the cancer is selected from breast, ovarian, pancreatic, or prostate cancer.

The invention also relates to a method of treatment of cancer in a patient in need thereof, wherein the method comprises the step of administering an effective amount of an inhibitor of the Bcl-2 family of proteins selected from navitoclax, venetoclax, A-1331852, AZD5991, or A-1155463 to the patient in combination with an effective amount of an aurora kinase inhibitor, wherein the cancer is selected from breast, ovarian, pancreatic, or prostate cancer.

The invention also relates to a method of treatment of cancer in a patient in need thereof, wherein the method comprises the step of administering an effective amount of an aurora kinase inhibitor to the patient in combination with an effective amount of an inhibitor of the Bcl-2 family of proteins selected from navitoclax, venetoclax, A-1331852, AZD5991, or A-1155463, wherein the cancer is selected from breast, ovarian, pancreatic, or prostate cancer.

The invention also relates to a use of a combination of an inhibitor of the Bcl-2 family of proteins selected from navitoclax, venetoclax, A-1331852, AZD5991, or A-1155463, together with an aurora kinase inhibitor in the manufacture of a medicament for the treatment of cancer in a patient, wherein the cancer is selected from breast, ovarian, pancreatic, or prostate cancer.

The invention also relates to a use of an inhibitor of the Bcl-2 family of proteins selected from navitoclax, venetoclax, A-1331852, AZD5991, or A-1155463 in the manufacture of a medicament for the treatment of cancer in a patient in combination with an aurora kinase inhibitor, wherein the cancer is selected from breast, ovarian, pancreatic, or prostate cancer.

The invention also relates to a use of an aurora kinase inhibitor in the manufacture of a medicament for the treatment of cancer in a patient in combination with an inhibitor of the Bcl-2 family of proteins selected from navitoclax, venetoclax, A-1331852, AZD5991, or A-1155463, wherein the cancer is selected from breast, ovarian, pancreatic, or prostate cancer.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows activity of combination therapies of the invention in 51 breast cancer cell lines, stratified by PAM50 sub-type, as described in Example 1. Combinations of navitoclax with AURK inhibitors are frequently synergistic in breast cancer cell lines, with exception of Her2 cells. Binary synergy for navitoclax (anchor) paired with three AURK inhibitors in cell lines from PAM50 subtypes.

FIG. 2 shows the sensitivity of breast cancer cell lines to Navitoclax+Aurora kinase inhibitor combinations. Breast cancer cell lines were selected to cover a range of PAM50 subtypes: Basal, Luminal A (LumA), Luminal B (LumB) or Her2. Cells were treated with Navitoclax plus an Aurora kinase inhibitor as indicated on each dot plot (A-G). The synergy metrics, Bliss window and I window, were generated as described. Each point is a single replicate, with 5-8 replicates per cell line.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Inhibitors of the Bcl-2 Family of Proteins

The Bcl-2 family of proteins consists of a number of evolutionarily-conserved proteins that share Bcl-2 homology (BH) domains. The Bcl-2 family is most notable for their regulation of apoptosis, a form of programmed cell death, at the mitochondrion. The Bcl-2 family proteins consists of members that either promote or inhibit apoptosis, and control apoptosis by governing mitochondrial outer membrane permeabilization (MOMP), which is a key step in the intrinsic pathway of apoptosis.

The Bcl-2 family of proteins comprises several family members, including Bcl-2 (B-cell lymphoma 2), Bcl-xL (B-cell lymphoma-extra large), Bcl-w (Bcl-2-like protein 2 or BCL2L2), Mcl-1 (induced myeloid leukemia cell differentiation protein), and A1 (Bcl-2-related protein A1 or BCL2A1).

The combination therapy described herein comprises an inhibitor of the Bcl-2 family of proteins. The inhibitor of the Bcl-2 family of proteins may be an inhibitor of one or more of the Bcl-2 family of proteins, such as Bcl-2, Bcl-xL, Bcl-w, Mcl-1, and A1, and combinations thereof. For example, inhibitor of the Bcl-2 family of proteins may be an inhibitor of Bcl-2 and Bcl-xL.

In some embodiments, the combination for use in the method of treatment described herein comprises an inhibitor of Bcl-2. That is, the combination for use in the method of treatment described herein comprises a Bcl-2 inhibitor.

In some embodiments, the combination for use in the method of treatment described herein comprises an inhibitor of Bcl-xL. That is, the combination for use in the method of treatment described herein comprises a Bcl-xL inhibitor.

In some embodiments, the combination for use in the method of treatment described herein comprises an inhibitor of Bcl-w. That is, the combination for use in the method of treatment described herein comprises a Bcl-w inhibitor.

In some embodiments, the combination for use in the method of treatment described herein comprises an inhibitor of Mcl-1. That is, the combination for use in the method of treatment described herein comprises a Mcl-1 inhibitor.

In some embodiments, the combination for use in the method of treatment described herein comprises an inhibitor of A1. That is, the combination for use in the method of treatment described herein comprises an A1 inhibitor.

Various Bcl-2, Bcl-xL, Bcl-w, Mcl-1, and A1 inhibitors are known in the art and include navitoclax, venetoclax, A-1331852, AZD5991, and A-1155463. In some embodiments, the combination for use in the method of treatment described herein comprises navitoclax. In some embodiments, the combination for use in the method of treatment described herein comprises venetoclax. In some embodiments, the combination for use in the method of treatment described herein comprises A-1331852. In some embodiments, the combination for use in the method of treatment described herein comprises AZD5991. In some embodiments, the combination for use in the method of treatment described herein comprises A-1155463.

Preferably, the combination for use in the method of treatment described herein comprises a Bcl-xL inhibitor, such as navitoclax, A-1331852, and A-1155463. In some embodiments, the combination for use in the method of treatment described herein comprises navitoclax. In some embodiments, the combination for use in the method of treatment described herein comprises A-1331852. In some embodiments, the combination for use in the method of treatment described herein comprises A-1155463.

### Navitoclax

Navitoclax, also referred to as ABT-263 or ABT263, is a Bcl-2 inhibitor (Kᵢ of ≤1 nM in cell-free assays). Navitoclax is also a potent inhibitor of Bcl-xL and Bcl-w with Kᵢ of ≤0.5 nM and ≤1 nM in cell-free assays, but binds more weakly to Mcl-1 and A1. Its structure is:

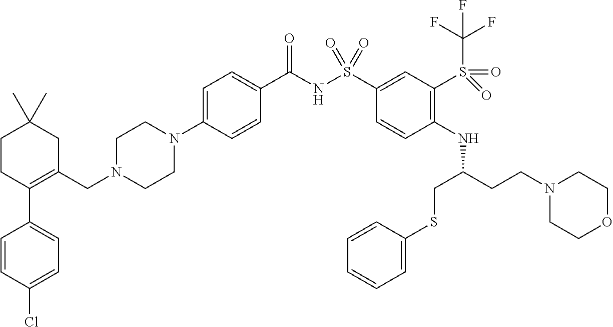 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

In IUPAC nomenclature, it may be referred to as 4-(4-{[2-(4-chlorophenyl)-5,5-dimethylcyclohex-1-en-1-yl]methyl}piperazin-1-yl)-N-(4-{[(2R)-4-(morpholin-4-yl)-1-(phenylsulfanyl) butan-2-yl]amino}-3-(trifluoromethanesulfonyl)benzene-1-sulfonyl)benzamide.

Navitoclax is disclosed in EP1888550, which document is incorporated herein by reference in its entirety, along with representative general synthetic methods. The compound is commercially available. Navitoclax is in Phase III clinical trials investigating the treatment of myelofibrosis.

### Venetoclax

Venetoclax is also known as ABT-199 and GDC-0199. It is a Bcl-2-selective inhibitor with Kᵢ of <0.01 nM in cell-free assays, >4800-fold more selective versus Bcl-xL and Bcl-w, and no activity to Mcl-1. Venetoclax is approved for the treatment of certain blood cancers. Its structure is:

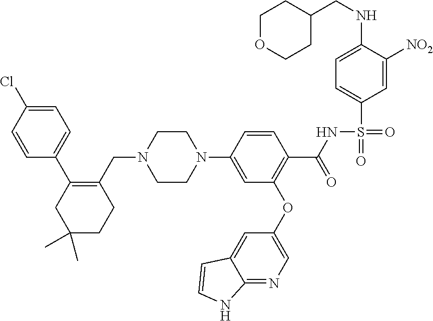 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

In IUPAC nomenclature, it may be referred to as 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide. Its synthesis is known in the art and the compound is commercially available.

### A-1331852

A-1331852 is a potent and selective Bcl-xL inhibitor with Kᵢ value less than 0.01 nM for Bcl-xL and 6 nM, 4 nM, 142 nM for Bcl-2, Bcl-w, and Mcl-1 respectively. It may be useful in the treatment of cancer, immune and autoimmune diseases. Its structure is:

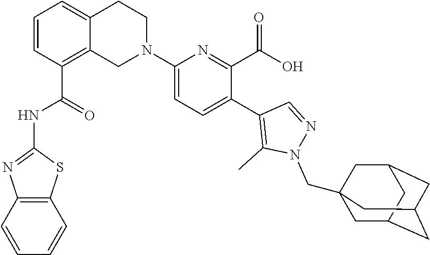 [SMILES: Cc1c(-c2ccc(N3CCc4cccc(C(=O)Nc5nc6ccccc6s5)c4C3)nc2C(=O)O)cnn1CC12CC3CC(CC(C3)C1)C2]

In IUPAC nomenclature, it may be referred to as 3-[1-(1-adamantylmethyl)-5-methyl-pyrazol-4-yl]-6-[8-(1,3-benzothiazol-2-ylcarbamoyl)-3,4-dihydro-1H-isoquinolin-2-yl]pyridine-2-carboxylic acid. Its synthesis is known in the art and the compound is commercially available.

### AZD5991

AZD5991 (or AZD-5991) is a macrocyclic Mcl-1 inhibitor with sub-nanomolar affinity for Mcl-1 (Kᵢ=0.13 nM). The binding affinity of AZD5991 is about 25-fold lower for mouse Mcl-1 vs. human Mcl-1 but only four-fold lower for rat Mcl-1. Its structure is:

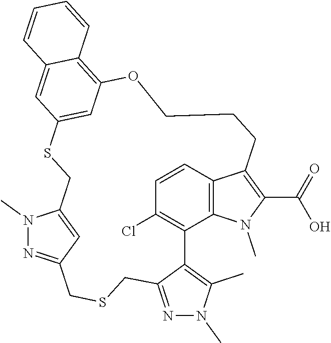 [SMILES: Cc1c2c(nn1C)CSCc1cc(n(C)n1)CSc1cc(c3ccccc3c1)OCCCc1c(C(=O)O)n(C)c3c-2c(Cl)ccc13]

In IUPAC nomenclature, it may be referred to as 17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30(35),31,33-tridecaene-23-carboxylic acid. Its synthesis is known in the art and the compound is commercially available.

AZD5991 exists in two rotamer forms or atropisomers (R) and(S) (conformational isomers that differ by rotation about a single bond):

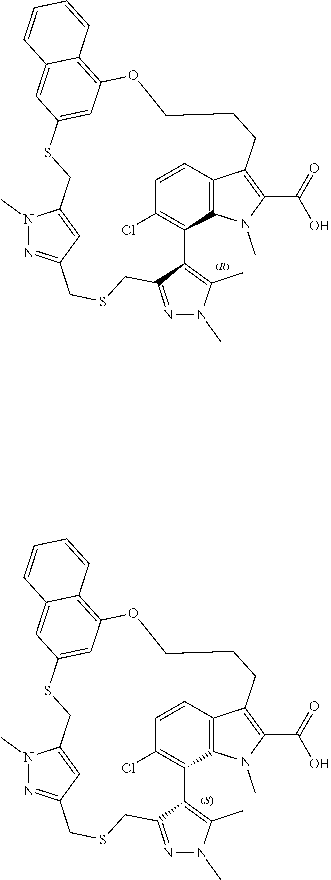

When referring to AZD5991 herein, we refer to both rotamers (or racemate) unless specifically specified.

### A-1155463

A-1155463 is a highly potent and selective Bcl-xL inhibitor. It shows picomolar binding affinity to Bcl-xL, and >1000-fold weaker binding to Bcl-2 and related proteins Bcl-w (Kᵢ=19 nM) and Mcl-1 (Kᵢ>440 nM). Its structure is:

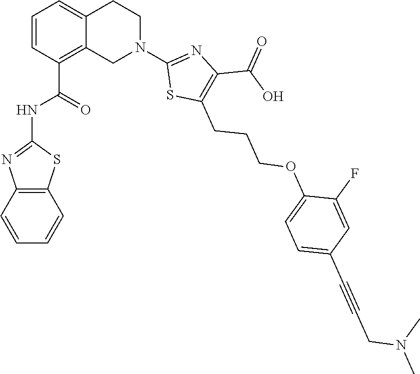 [SMILES: CN(C)CC#Cc1ccc(OCCCc2sc(N3CCc4cccc(C(=O)Nc5nc6ccccc6s5)c4C3)nc2C(=O)O)c(F)c1]

In IUPAC nomenclature, it may be referred to as 2-[8-(1,3-benzothiazol-2-ylcarbamoyl)-3,4-dihydro-1H-isoquinolin-2-yl]-5-[3-[4-[3-(dimethylamino) prop-1-ynyl]-2-fluoro-phenoxy]propyl]thiazole-4-carboxylic acid. Its synthesis is known in the art and the compound is commercially available.

### Aurora Kinase Inhibitors

Aurora kinase inhibitors are also referred to herein and in the art as AURK inhibitors as AURKi.

Aurora kinases (AURK) are phosphotransferase enzymes understood to be important in cellular division processes. Modulation of the aurora kinase pathways is of interest for the treatment of cancer, and in particular in the prevention and treatment of tumorigenesis.

Three classes of AURK have been identified in humans: Aurora A, Aurora B and Aurora C. Aurora A is also known as Aurora 2 and Aurora B is also known as Aurora 1.

In some embodiments, the AURK inhibitors of the present invention inhibits Aurora A kinase.

Various AURK inhibitors are known in the art and include alisertib, tozasertib, ZM447439, AZD2811 (the active barasertib-hQPA), AZD1152 (barasertib, a pro-drug of barasertib-hQPA), LY3295668, MK-5108, GSK1070916, and MLN8054. In some embodiments, the aurora kinase inhibitor is selected from alisertib, tozasertib, ZM447439, AZD2811, and AZD1152. In some embodiments, the aurora kinase inhibitor is selected from alisertib, tozasertib, and ZM447439. In some embodiments, the AURKi is alisertib. In some embodiments, the AURKi is tozasertib. In some embodiments the AURKi is ZM447439.

### Alisertib

Alisertib is also known as MLN8237 and is an orally available selective aurora A kinase inhibitor. Its structure is:

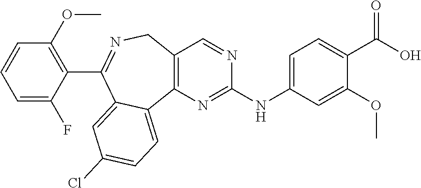 [SMILES: COc1cc(Nc2ncc3c(n2)-c2ccc(Cl)cc2C(c2c(F)cccc2OC)=NC3)ccc1C(=O)O]

In IUPAC nomenclature, it may be referred to as 4-{[9-chloro-7-(2-fluoro-6-methoxyphenyl)-5H-pyrimido[5,4-d][2]benzazepin-2-yl]amino}-2-methoxybenzoic acid. Its synthesis is known in the art and the compound is commercially available.

### Tozasertib

Tozasertib, also known as VX-680 and MK-0457, is a pan-aurora kinase inhibitor. It is much more potent against aurora A kinase than B/C. Its structure is:

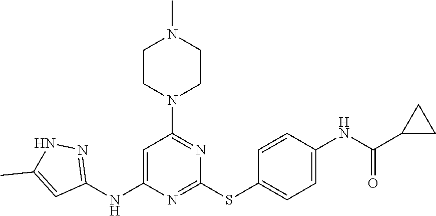 [SMILES: Cc1cc(Nc2cc(N3CCN(C)CC3)nc(Sc3ccc(NC(=O)C4CC4)cc3)n2)n[nH]1]

In IUPAC nomenclature, it may be referred to as N-[4-({4-(4-methylpiperazin-1-yl)-6-[(5-methyl-1H-pyrazol-3-yl)amino]pyrimidin-2-yl}sulfanyl)phenyl]cyclopropanecarboxamide. Its synthesis is known in the art and the compound is commercially available.

### ZM447439

ZM447439 is a selective and ATP-competitive inhibitor for aurora A kinase and aurora B kinase. Its structure is:

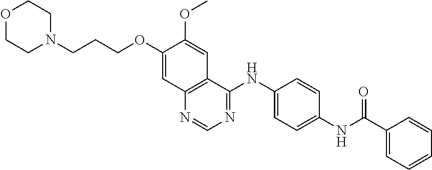 [SMILES: COc1cc2c(Nc3ccc(NC(=O)c4ccccc4)cc3)ncnc2cc1OCCCN1CCOCC1]

In IUPAC nomenclature, it may be referred to as N-[4-({6-methoxy-7-[3-(morpholin-4-yl) propoxy]quinazolin-4-yl}amino)phenyl]benzamide. Its synthesis is known in the art and the compound is commercially available.

### AZD2811

AZD2811 is a selective aurora B kinase inhibitor with an IC₅₀ of 0.37 nM in a cell-free assay. Also known as barasertib-hQPA (-hydroxyquinazoline-pyr-azol-aniline) or AZD1152-hQPA, it is the active metabolite of AZD1152 described below. AZD2811 induces growth arrest and apoptosis in cancer cells. Its structure is:

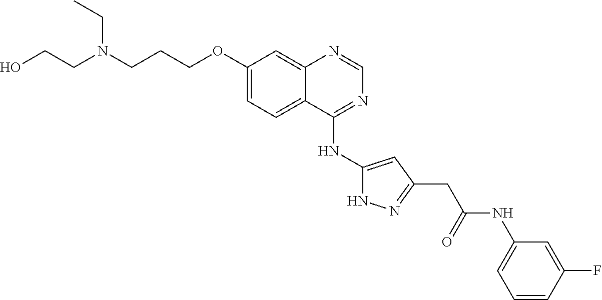 [SMILES: CCN(CCO)CCCOc1ccc2c(Nc3cc(CC(=O)Nc4cccc(F)c4)n[nH]3)ncnc2c1]

In IUPAC nomenclature, it may be referred to as 2-[3-[[7-[3-[ethyl(2-hydroxyethyl)amino]propoxy]quinazolin-4-yl]amino]-1H-pyrazol-5-yl]-N-(3-fluorophenyl) acetamide. Its synthesis is known in the art and the compound is commercially available. AZD2811 may be provided as an Accurin™ formulation (BIND Therapeutics, Inc.).

### AZD1152

AZD1152 is also known as barasertib. AZD1152 is a pro-drug of barasertib-hQPA (AZD2811), which is a highly selective aurora B kinase inhibitor with an IC₅₀ of 0.37 nM in a cell-free assay. Its structure is:

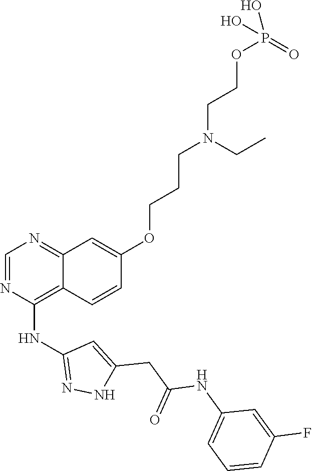 [SMILES: CCN(CCCOc1ccc2c(Nc3cc(CC(=O)Nc4cccc(F)c4)[nH]n3)ncnc2c1)CCOP(=O)(O)O]

In IUPAC nomenclature, it may be referred to as 2-[ethyl-[3-[4-[[5-[2-(3-fluoroanilino)-2-oxoethyl]-1H-pyrazol-3-yl]amino]quinazolin-7-yl]oxypropyl]amino]ethyl dihydrogen phosphate. Its synthesis is known in the art and the compound is commercially available.

### LY3295668

LY3295668 is also known as AK-01. It is a potent, orally active and specific inhibitor of Aurora A kinase with Kᵢ of 0.8 nM and 1038 nM for AURKA and AURKB, respectively. Its structure is:

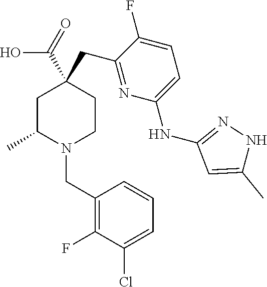

In IUPAC nomenclature, it may be referred to as (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid. Its synthesis is known in the art and the compound is commercially available.

LY3295668 may also exist as other isomeric forms (2R,4S), (2S,4R), and (2S,4S). That is, LY3295668 may also exist as (2R,4S)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid; (2S,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid; and (2S,4S)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid.

When referring to LY3295668 herein, we refer to all isomeric forms unless specifically specified.

### MK-5108

MK-5108 is also known as VX-689. It is a highly selective Aurora A kinase inhibitor with IC₅₀ of 0.064 nM in a cell-free assay. It is 220- and 190-fold more selective for Aurora A kinase than Aurora B/C, while it inhibits Tropomyosin receptor kinase A (TrkA) with less than 100-fold selectivity. MK-5108 is known to induce autophagy. It has the following structure:

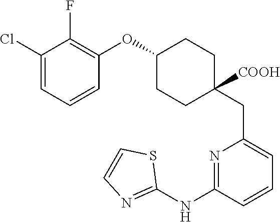 [SMILES: O=C(O)[C@]1(Cc2cccc(Nc3nccs3)n2)CC[C@@H](Oc2cccc(Cl)c2F)CC1]

In IUPAC nomenclature, it may be referred to as (4-(3-chloro-2-fluoro-phenoxy)-1-[[6-(thiazol-2-ylamino)-2-pyridyl]methyl]cyclohexanecarboxylic acid. Its synthesis is known in the art and the compound is commercially available.

### GSK1070916

GSK1070916 is a reversible and ATP-competitive inhibitor of Aurora B/C kinase with IC₅₀ of 3.5 nM/6.5 nM. It displays >100-fold selectivity against the closely related Aurora A-TPX2 complex. It has the following structure:

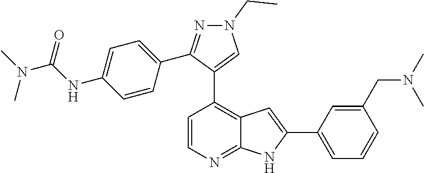

In IUPAC nomenclature, it may be referred to as 3-[4-[4-[2-[3-[(dimethylamino)methyl]phenyl]-1H-pyrrolo[2,3-b]pyridin-4-yl]-1-ethyl-pyrazol-3-yl]phenyl]-1,1-dimethyl-urea. Its synthesis is known in the art and the compound is commercially available.

### MLN8054

MLN8054 is a potent and selective inhibitor of Aurora A kinase with IC₅₀ of 4 nM in Sf9 insect cell. It is more than 40-fold selective for Aurora A kinase than Aurora B kinase. It has the following structure:

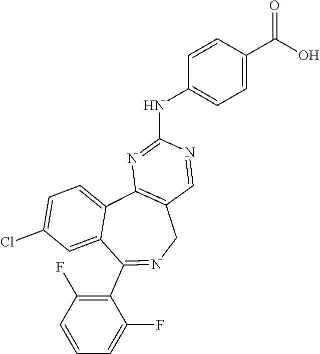 [SMILES: O=C(O)c1ccc(Nc2ncc3c(n2)-c2ccc(Cl)cc2C(c2c(F)cccc2F)=NC3)cc1]

In IUPAC nomenclature, it may be referred to as 4-[[9-chloro-7-(2,6-difluorophenyl)-5H-pyrimido[5,4-d][2]benzazepin-2-yl]amino]benzoic acid. Its synthesis is known in the art and the compound is commercially available.

### Specific Inhibitor Combinations

In some cases, the combination of an inhibitor of Bcl-2 family of proteins, such as Bcl-2, Bcl-xL, Bcl-w, Mcl-1, and A1 inhibitors, together with an aurora kinase inhibitor for use in a method of treatment described herein may be specific inhibitor combinations.

In some cases, the combination may be selected from: navitoclax and alisertib; navitoclax and barasertib; navitoclax and GSK1070916; navitoclax and LY3295668; navitoclax and MLN8054; navitoclax and tozasertib; navitoclax and ZM447439; A-1331852 and alisertib; A-1331852 and LY3295668; A-1331852 and MK-5108; A-1155463 and alisertib; A-1155463 and LY3295668; and A-1155463 and MK-5108.

In some cases, the combination may be selected from: navitoclax and alisertib; navitoclax and barasertib; navitoclax and GSK1070916; navitoclax and LY3295668; navitoclax and MLN8054; navitoclax and tozasertib; navitoclax and ZM447439; and A-1331852 and alisertib.

In some cases, the combination for use in a method of treatment described herein may be the combination of navitoclax and alisertib. In some cases, the combination for use in a method of treatment described herein may be the combination of navitoclax and barasertib. In some cases, the combination for use in a method of treatment described herein may be the combination of navitoclax and GSK1070916. In some cases, the combination for use in a method of treatment described herein may be the combination of navitoclax and LY3295668. In some cases, the combination for use in a method of treatment described herein may be the combination of navitoclax and MLN8054. In some cases, the combination for use in a method of treatment described herein may be the combination of navitoclax and tozasertib. In some cases, the combination for use in a method of treatment described herein may be the combination of navitoclax and ZM447439. In some cases, the combination for use in a method of treatment described herein may be the combination of A-1331852 and alisertib.

### Pharmaceutically Acceptable Salts

As described herein, any compound may be provided as a pharmaceutically acceptable salt, hydrate or solvate. Suitable pharmaceutically acceptable salts are known in the art and are described in, for example, in Berge et al., J Pharm Sci, 1977 66(1) p 1.

### Administration of Active Ingredients

Compounds used in the methods of the invention may be administered by any suitable route, including oral and intravenous routes. It will be understood that oral administration may be preferred. The compounds may be provided in pharmaceutical compositions comprising the compound and one or more pharmaceutically acceptable excipients. Formulation for oral administration may be in the form of a tablet or a capsule comprising a powder or liquid.

Administration is preferably in a “therapeutically effective amount” or an “effective amount” (used interchangeably), this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20^{th} Edition, 2000, pub. Lippincott, Williams & Wilkins.

Dosage regimens known in the art for the active ingredients described herein may also be used in the present invention.

For example, navitoclax has been administered as a single agent at a dose of 325 mg orally in a continuous 21/21 dosing schedule (Wilson et al., Lancet Oncol.; 2010 December; 11 (12): 1149-1159). When combined with 1000 mg/m² of gemcitabine, Navitoclax was also administered as 325 mg in Cleary et al. (Invest New Drugs, 2014 October; 32 (5): 937-945). Navitoclax has also been administered intermittently over a 21-day cycle, wherein the patients on the intermittent dosing regimen received navitoclax on day −3, followed by dosing on days 1 to 14 of a 21-day cycle. Patients in the continuous dosing regimen received a 1-week lead-in dose of 150 mg followed by continuous daily administration in the same study (Gandhi et al., J Clin Oncol, 2011 Mar. 1; 29 (7): 909-916). It will be appreciated therefore that a suitable dosage regimen for navitoclax may be prescribed by a clinician. Suitable dosage regimens for venetoclax, A-1331852, AZD5991, and A-1155463 are also described in the art.

Dosage regimens of AURKi, such as alisertib, tozasertib, ZM447439, AZD2811, AZD1152 and LY3295668, are also known in the art. For example, alisertib has been tested in a Phase I clinical study in East Asian patients with advanced solid tumours or lymphomas. The patients received alisertib twice daily (BID) for 7 days in 21-day cycles, where the doses were escalated from 30 mg BID to 40 mg BID, which was well-tolerated (Venkatakrishnan et al., Invest New Drugs, 2015 August; 33 (4): 942-953). This is slightly lower than the 50 mg BID dosage of alisertib received by Western patients (Beltran et al., Clin. Cancer Res., 2019 Jan. 1; 25 (1): 43-51; Kelly et al., Invest New Drugs, 2014 June; 32 (3): 489-499).

Tozasertib (also known as MK-0457 or VX-680) has also been tested in a Phase I dose escalation study in adult patients with advanced solid tumours. The patients were dosed with an escalating 24-hour continuous intravenous (CIV) infusion of tozasertib every 21 days, with the maximum tolerated dose (MTD) reaching 64 mg/m²/hr. The patients were also dosed with a 100 mg of tozasertib orally (Traynor et al., Cancer Chemother Pharmacol. 2011 February; 67 (2): 305-314). In a Phase II study, the patients were dosed with a 5-day continuous infusion of tozasertib every 14 days at 40 mg/m²/h, 32 mg/m²/h or 24 mg/m²/h (Seymour et al., Blood Cancer Journal, 2014, 4, e238). Suitable dosage regimens for ZM447439, AZD2811, AZD1152, and LY3295668 are also described in the art.

Accordingly, the active ingredients described herein may be administered in dosages of about 1 mg to about 1000 mg, such as about 5 mg to about 500 mg, such as about 10 mg to about 400 mg.

In some embodiments, navitoclax may be administered in a dosage of about 1 mg to about 1000 mg, such as about 5 mg to about 500 mg, such as about 10 mg to about 400 mg, such as about 150 mg to about 325 mg, such as about 150 mg or about 325 mg. In some embodiments, venetoclax may be administered in a dosage of about 1 mg to about 1000 mg, such as about 5 mg to about 500 mg, such as about 10 mg to about 400 mg, such as about 150 mg to about 325 mg, such as about 150 mg or about 325 mg. In some embodiments, A-1331852 may be administered in a dosage of about 1 mg to about 1000 mg, such as about 5 mg to about 500 mg, such as about 10 mg to about 400 mg, such as about 150 mg to about 325 mg, such as about 150 mg or about 325 mg. In some embodiments, AZD5991 may be administered in a dosage of about 1 mg to about 1000 mg, such as about 5 mg to about 500 mg, such as about 10 mg to about 400 mg, such as about 150 mg to about 325 mg, such as about 150 mg or about 325 mg. In some embodiments, A-1155463 may be administered in a dosage of about 1 mg to about 1000 mg, such as about 5 mg to about 500 mg, such as about 10 mg to about 400 mg, such as about 150 mg to about 325 mg, such as about 150 mg or about 325 mg.

In some embodiments, AURKi, such as alisertib, tozasertib, ZM447439, AZD2811, AZD1152 and LY3295668, may be administered in a dosage of about 1 mg to about 1000 mg, such as about 5 mg to about 500 mg, such as about 10 mg to about 300 mg, such as about 20 mg to about 100 mg, such as about 30 mg to about 50 mg, such as about 30 mg or about 40 mg or about 50 mg.

The active ingredients described herein may be administered simultaneously or consecutively, or at different times within a prescribed dosing cycle. The active ingredients described herein may be administered daily, such as once daily (QD), twice daily (BID), three times daily (TID), or four times daily (QID), or on a less frequent or intermittent schedule.

Suitably, the patient may be a human patient.

### Cancer Type

The invention relates to methods for the treatment of cancer in patients, and in particular basal-like, TNBC (triple negative breast cancer), and/or HRD (homologous recombination deficiency) cancers.

The diagnosis of HRD deficiency is recognised in the art and can be based on of criteria including:
1. Genetic testing for somatic or germline loss of function mutation of BRCA1 or BRCA2 genes are used clinically to define HRD. See, for example, González-Martin et al. N Engl J Med 2019; 381 (25): 2391-2402 (PMID 51362799). Available testing platforms include myChoice® CDx from Myriad Genetics, Inc. (https://myriad.com/products-services/precision-medicine/mychoice-cdx/), which has been FDA-approved as a companion diagnostic for Lynparza™ (olaparib) for advanced ovarian cancer (https://myriad.com/investors/news-release/news-release-detail/?newsItemId=21171), and FoundationOneLiquid® CDx from Foundation Medicine, Inc. (https://www.foundationmedicine.com/test/foundationone-liquid-cdx) which has been FDA approved (https://www.fda.gov/drugs/resources-information-approved-drugs/fda-grants-marketing-approval-foundationone-cdx-in-vitro-diagnostic). Information about the FoundationOne® CDx platform can be found at https://assets.ctfassets.net/w98cd481qyp0/41rJj28gFwtxCwHQxopaEb/fba378cd309082f09570f32fc16b5d01/FoundationOne_CDx_Label_Technical_Info.pdf, for example.
2. Mutational signatures analysis from whole genome sequencing data have been proposed. These assays look for ‘genetic scars’ as a consequence of HRD. One type looks for changes in gene copy number profiles and the other for patterns of nucleotide substitutions. The weighted model HRDetect has also been developed, which predicts germline or somatic mutation of BRCA1 or BRCA2, as well as functional BRCA1 or BRCA2 deficiency where no mutation is detected, based on mutational signatures (Davies et al., Nat Med. 2017 April; 23 (4): 517-525).
3. Alternations in other DNA repair pathways genes can also confer HRD (e.g. ATM, ATR, PALB2, RAD51C, RAD51D, BRIP1, BARD1, RPA, PTEN, CHEK1, CHEK2, MRE11, RADa50, MLH1, MSH2, MSH6, PMS2 . . . etc [this list is not comprehensive])
4. Functional assays such as RAD51 foci formation in cells, or sensitivity to platinum-based drugs (Ngoi and Tan, ESMO Open, 2021 June; 6 (3): 100144).

In some embodiments, the cancer has been determined to be HRD deficient by one or more the above criteria or tests.

A defining feature of TNBC is an increased frequency of BRCA1/2 mutations. Its diagnosis is recognised in the art.

In some cases, the treatment is of a cancer selected from breast cancer, ovarian cancer, pancreatic cancer, and prostate cancer. Breast cancer, ovarian cancer, pancreatic cancer and prostate cancer are cancer types in which there is a proportion of tumours that are HRD, and therefore the inventors postulate that treatments which are effective in HER2-negative/TNBC might also be effective in ovarian cancer, pancreatic cancer and prostate cancer. By example, PARP inhibitors are used clinically to treat breast cancer, ovarian cancer, prostate cancer and pancreatic cancer patients with BRCA1/2 mutant tumours.

The patient may have breast cancer. Breast cancer is a heterogeneous disease typically categorized into five subtypes: luminal A (LumA), luminal B (LumB), HER2-enriched (HER2 or HER2-E), basal-like (basal) and normal breast-like (normal). These subtypes can be distinguished based on the expression level of four recognised biomarkers by immunohistochemistry: estrogen receptor (ER), progesterone receptor (PR), human epidermal growth factor receptor 2 (HER2), and Ki-67. For example, the National Cancer Institute provides information on Breast Cancer Treatment (Adult) (PDQ®)—Health Professional Version which can be accessed here: https://www.cancer.gov/types/breast/hp/breast-treatment-pdq #_18. It will be appreciated that other advice and resources are available to clinicians and the skilled person, but that the determination of breast cancer type is routine and well-understood in the art.

Suitably, the breast cancer is non-HER2-enriched (non-HER2-E) breast cancer. That is, the breast cancer may be categorized as luminal A (LumA), luminal B (LumB), or basal-like (basal). Diagnostically, HER2 status is typically defined by IHC/FISH subtyping. IHC refers to immunohistochemistry test and FISH refers to fluorescence in situ hybridization test. IHC for HER2 is categorised as 0, +1, +2 or +3, with a result of +3 indicating HER2-enriched cancer and a result of 0 or 1+ indicating non-HER2-enriched cancer. A result of +2 is consider equivocal, in which case FISH is normally also used.

Accordingly, the invention may relate to the treatment of non-HER2-enriched breast cancer as determined by IHC and/or FISH subtyping in a patient.

Alternatively, the invention may relate to the treatment of non-HER2-enriched breast cancer as determined by molecular subtyping in a patient based on gene expression, such as PAM50 subtyping in a patient. The molecular subtyping in a patient based on gene expression, such as PAM50 subtyping, may be determined by gene expression analysis, such as using a diagnostic test like Oncotype DX®.

In some cases, the breast cancer is basal-like.

In some cases, the breast cancer is triple negative breast cancer (TNBC). The TNBC may have a basal-like gene express or a non-basal like gene expression. Its diagnosis is recognised in the art and described, for example, in information provided by the National Cancer Institute (Breast Cancer Treatment (Adult) (PDQ®)—Health Professional Version) which can be accessed here: https://www.cancer.gov/types/breast/hp/breast-treatment-pdq #_18.

In some cases, the breast cancer is luminal A.

In some cases, the breast cancer is luminal B.

### PAM50 Subtyping

Patients described herein may be categorised using PAM50 subtyping. PAM50 is a 50-gene test that is designed to identify intrinsic breast cancer subtypes (Basal, Her2, LumA, LumB, and Normal) and generate a Risk of Recurrence (ROR) score and was developed to be carried out in qualified routine hospital pathology laboratories. It is described in Gnant et al. Ann. Oncol.; 2014 25 (2) pp. 339-45 and Nielson et al. Clin Cancer Res; 2010 16 (21) pp. 5255-32.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1

Navitoclax was screened in combination with three AURK inhibitors (alisertib, ZM447439, tozasertib) in 51 breast cancer cell lines. Viability was read out after 72 h of drug treatment using CellTiter-Glo and drug responses for single agent and combination responses were fitted.

Synergy was called based on significant shifts in efficacy (ΔEₘₐₓ) or potency (ΔIC₅₀) of the combination over the expected drug combination response based on their single agent activities [as calculated by Bliss (Bliss, Annals of Applied Biology, 1939, 26 (3), 585-615); synergy if ΔEₘₐₓ≥20% viability or ΔIC₅₀≥3 (=8-fold concentration shift)]. Activity was called based on significant shifts of viability reduction of the combination compared to single agent activity (for example, for efficacy if the combination showed 20% more viability reduction than the predicted effect of the combination based on the single agent activity).

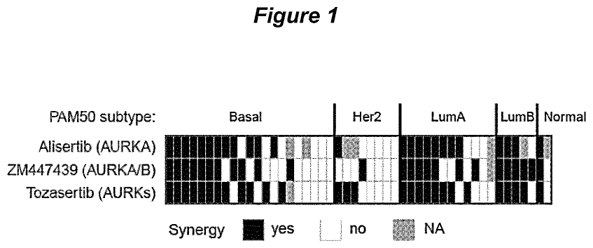

Using RNASeq data, breast cancer cell lines were annotated for PAM50 subtype. See FIG. 1.

Navitoclax combined with any of the three aurora kinase (AURK) inhibitors alisertib, tozasertib or ZM447439 had particularly high synergy rates in the cell lines (61%, 60% and 53% of cell lines, respectively).

Navitoclax with alisertib and ZM447439 was included in an independent validation screen performed using the same format and assay, and synergy was reproducible (94% and 88% overlap of cell line synergy calls between screens, respectively).

Synergy was frequently observed for at least two out of three navitoclax and AURKi combinations in cell lines from all PAM50 subtypes (63% (12/19 cell lines) in basal-like, 73% (8/11) in LumA and 75% (3/4) in LumB), with the exception of Her2 cell lines (17% or 1/6 cell lines) where the frequency of synergy was much lower. See Table 1 and FIG. 1.

TABLE 1

| Synergy in 2/3 | | | | |
|---|---|---|---|---|
| combinations | Basal | Her2 | LumA | LumB |
| % | 63% | 17% | 73% | 75% |
| # of cell lines | 12/19 | 1/6 | 8/11 | 3/4 |

### Example 2

### Other Navitoclax+Aurora Kinase Inhibitor Combinations in Cancer Cell Lines

### Method:

A screen was carried out in 10 breast cancer cell lines (JIMT-1, HCC38, BT-20, CAL-51, CAMA-1, HCC1428, BT-483, MDA-MB-415, EFM-192A, HCC1419) using a 7×7 matrix approach generating 49 wells of data per cell line/drug combination. For each combination, one Aurora kinase inhibitor was combined with Navitoclax, over a discontinuous 1,000-fold (7-point) dose range. Viability was measured after 72 h of drug treatment using CellTiter-Glo reagent. Single-agent and combination viability measurements were fitted per cell line and multiple parameters derived including single agent values and a range of synergy scores.

For all 49 concentration combination measurements a Bliss excess was calculated by comparing the observed combination response of cells to the Bliss independence-predicted response based on monotherapy activity. The “Bliss window” was reported as the highest mean Bliss excess value measured across the 25 possible 3×3 submatrices, or ‘windows’, across the 7×7 dose matrix.

In addition, a I (Highest Single Agent) excess was calculated for all 49 concentration combination measurements by comparing the observed combination response of cells to the highest single agent response for either Drug A or Drug B, whichever is highest. The “I window” was reported as the highest mean I excess value measured across the 25 possible 3×3 submatrices, or ‘windows’, across the 7×7 dose matrix.

### Results:

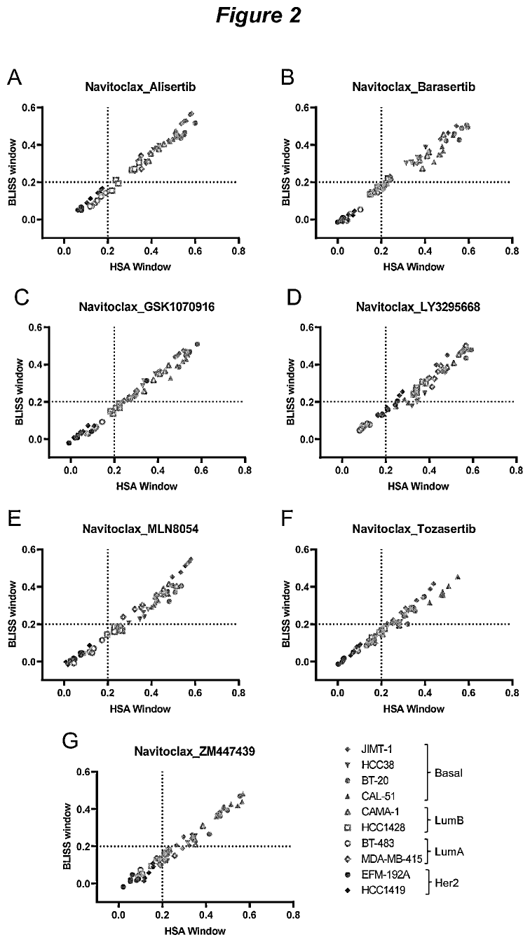

This screen comprised Navitoclax in combination with each of the following Aurora kinase inhibitors: Alisertib, MLN8054, Barasertib, Tozasertib, GSK1070916, ZM447439, LY3295668. The cell lines used covered a range of PAM50 subtypes including two annotated as Her2 (FIG. 2A-G). Results show that synergy with all combinations is low in the Her2 cell lines as judged by the presence of this PAM50 subtype in the lower left of the plots in FIG. 2A-G. In contrast, most of the Basal, Luminal A and Luminal B cell lines show greater synergy in response to all combinations, as seen from the higher Bliss window and I window scores.

### Example 3

### Other BH3 Family and Aurora Kinase Inhibitor Combinations in Cancer Cell Lines

### Method:

Compounds were tested in the Her2 positive breast cancer cell line, JIMT-1, using a 7×7 matrix approach generating 49 wells of data per cell line/drug combination. For each combination, Alisertib was combined with a single BH3 mimetic, over a discontinuous 1,000-fold (7-point) dose range. Viability was measured after 72 h of drug treatment using CellTiter-Glo reagent. Single-agent and combination viability measurements were fitted per cell line and multiple parameters derived including single agent values and a range of synergy scores.

Single-agent and combination viability measurements were fitted per for each combination and multiple parameters derived including: 1) single agent viability effect, 2) single agent and combination viability effect at the highest-used concentration (Eₘₐₓ), and 3) the estimated drug concentration producing a 50% viability reduction (IC₅₀) for the single agents and combination. We compared observed combination response of cells to the Bliss independence-predicted response based on monotherapy activity, and classified drug combinations based on shifts beyond Bliss independence (Bliss, Annals of Applied Biology, 1939, 26 (3), 585-615) in potency (ΔIC₅₀; i.e. increased sensitivity) or efficacy (ΔEₘₐₓ; i.e. reduced cell viability).

### Results:

Two different inhibitors of Bcl-2 family proteins were tested in combination with the Aurora kinase inhibitor, Alisertib in JIMT-1 cells, a Her2 positive breast cancer cell line. The Bcl-2 family can be divided into pro-apoptotic proteins (including BAX and BAK) and anti-apoptotic proteins (including Bcl-2, Bcl-xL and Mcl-1). All anti-apoptotic Bcl-2 family proteins share a BH3 domain, a structural feature which has been exploited in the treatment of cancer for the development of a class of drugs known as BH3 mimetics. These drugs mimic the activity of the pro-apoptotic proteins and inhibit other pro-survival family members (Diepstraten et al., Nat Rev Cancer. 2022 January; 22 (1): 45-64; Kelekar and Thompson Trends Cell Biol. 1998 August; 8 (8): 324-30; Townsend et al., J Exp Clin Cancer Res. 2021 Nov. 9; 40 (1): 355). Navitoclax is a potent inhibitor of Bcl-2, Bcl-xL and Bcl-w but binds more weakly to Mcl1 and A1, while A-1331852 is a Bcl-xL selective inhibitor, showing ≥400-fold selectivity for Bcl-xL compared to Bcl-2, Bcl-w and Mcl-1. Table 2 below shows the maximum values achieved for ΔIC₅₀ and ΔEₘₐₓ for each combination, from two independent replicates. The results show that both Navitoclax and A-1331852 are synergistic with Alisertib in this cell line.

TABLE 2

| | | Replicate 1 | | Replicate 2 | |
|---|---|---|---|---|---|
| | | Log₂ | | Log₂ | |
| BH3 mimetic | AURKi | ΔIC₅₀ | ΔEₘₐₓ | ΔIC₅₀ | ΔEₘₐₓ |
| Navitoclax | Alisertib | 3.7 | 0.38 | 3.6 | 0.39 |
| A-1331852 | Alisertib | 3.6 | 0.31 | 2.5 | 0.43 |

### REFERENCES

A number of publications are cited above in order to more fully describe and disclose the invention and the state of the art to which the invention pertains. Full citations for these references are provided below. The entirety of each of these references is incorporated herein.
Beltran et al., Clin. Cancer Res., 2019 Jan. 1; 25 (1): 43-51
Berge et al., J Pharm Sci, 1977 66 (1) p 1
Bliss, Annals of Applied Biology, 1939, 26 (3), 585-615
Cleary et al., Invest New Drugs, 2014 October; 32 (5): 937-945
Davies et al., Nat Med. 2017 April; 23 (4): 517-525
Diepstraten et al., Nat Rev Cancer. 2022 January; 22 (1): 45-64
EP1888550
Gandhi et al., J Clin Oncol, 2011 Mar. 1; 29 (7): 909-916
Gnant et al. Ann. Oncol.; 2014 25 (2) pp. 339-45
González-Martin et al. N Engl J Med 2019; 381 (25): 2391-2402 (PMID 51362799)
Kelekar and Thompson Trends Cell Biol. 1998 August; 8 (8): 324-30
Kelly et al., Invest New Drugs, 2014 June; 32 (3): 489-499
Ngoi and Tan, ESMO Open, 2021 June; 6 (3): 100144
Nielson et al. Clin Cancer Res; 2010 16 (21) pp. 5255-32
Seymour et al., Blood Cancer Journal, 2014, 4, e238
Townsend et al., J Exp Clin Cancer Res. 2021 Nov. 9; 40 (1): 355
Traynor et al., Cancer Chemother Pharmacol. 2011 February; 67 (2): 305-314
Wilson et al., Lancet Oncol.; 2010 December; 11 (12): 1149-1159
Venkatakrishnan et al., Invest New Drugs, 2015 August; 33 (4): 942-953
https://assets.ctfassets.net/w98cd481qyp0/41rJj28gFwtxCwHQxopaEb/fba378cd309082f09570f32fc16b5d01/FoundationOne_CDx_Label_Technical_Info.pdf
https://www.cancer.gov/types/breast/hp/breast-treatment-pdq #_18
https://myriad.com/products-services/precision-medicine/mychoice-cdx/

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

### Priority Clauses

1. A combination of navitoclax or venetoclax together with an aurora kinase inhibitor for use in a method of treatment of a cancer selected from breast, ovarian, pancreatic, or prostate cancer in a patient.
2 The combination for use according to clause 1, wherein the combination is navitoclax and an aurora kinase inhibitor.
3. The combination for use according to clause 1, wherein the combination is venetoclax and an aurora kinase inhibitor.
4. The combination for use according to any preceding clause, wherein the cancer is non-HER2− enriched breast cancer.
5. The combination for use according to any preceding clause, wherein the cancer is determined to be HER2−.
6. The combination for use according to any preceding clause, wherein the cancer is basal-like breast cancer.
7. The combination for use according to any preceding clause, wherein the cancer is triple negative breast cancer.
8. The combination for use according to any of clauses 1 to 5, wherein the cancer is luminal B breast cancer.
9. The combination for use according to any of clauses 1 to 5, wherein the cancer is luminal A breast cancer.
10. The combination for use according to any preceding clause, wherein the aurora kinase inhibitor is an aurora A kinase inhibitor.
11. The combination for use according to any preceding clause, wherein the aurora kinase inhibitor is selected from alisertib, tozasertib, ZM447439, AZD2811, and AZD1152.
12. The combination for use according to any preceding clause, wherein the aurora kinase inhibitor is selected from alisertib, tozasertib, and ZM447439.
13. The combination for use according to any preceding clause, wherein navitoclax or venetoclax and the aurora kinase inhibitor are administered separately.

1. A combination of an inhibitor of the Bcl-2 family of proteins selected from navitoclax, venetoclax, A-1331852, AZD5991, or A-1155463, together with an aurora kinase inhibitor for use in a method of treatment of a cancer selected from breast, ovarian, pancreatic, or prostate cancer in a patient.

2. An inhibitor of the Bcl-2 family of proteins selected from navitoclax, venetoclax, A-1331852, AZD5991, or A-1155463, for use in a method of treatment of a cancer selected from breast, ovarian, pancreatic, or prostate cancer in a patient, and wherein the inhibitor of the Bcl-2 family of proteins is administered to the patient in combination with an aurora kinase inhibitor.

3. An aurora kinase inhibitor for use in a method of treatment of a cancer selected from breast, ovarian, pancreatic, or prostate cancer in a patient, and wherein the aurora kinase inhibitor is administered to the patient in combination with an inhibitor of the Bcl-2 family of proteins selected from navitoclax, venetoclax, A-1331852, AZD5991, or A-1155463.

4. The combination for use according to claim 1, the inhibitor of the Bcl-2 family of proteins for use according to claim 2, or the aurora kinase inhibitor for use according to claim 3, wherein the inhibitor of the Bcl-2 family of proteins is navitoclax.

5. The combination for use according to claim 1, the inhibitor of the Bcl-2 family of proteins for use according to claim 2, or the aurora kinase inhibitor for use according to claim 3, wherein the inhibitor of the Bcl-2 family of proteins is A-1331852.

6. The combination for use according to claim 1, the inhibitor of the Bcl-2 family of proteins for use according to claim 2, or the aurora kinase inhibitor for use according to claim 3, wherein the inhibitor of the Bcl-2 family of proteins is A-1155463.

7. The combination for use according to claim 1, the inhibitor of the Bcl-2 family of proteins for use according to claim 2, or the aurora kinase inhibitor for use according to claim 3, wherein the inhibitor of the Bcl-2 family of proteins is venetoclax.

8. The combination for use according to claim 1, the inhibitor of the Bcl-2 family of proteins for use according to claim 2, or the aurora kinase inhibitor for use according to claim 3, wherein the inhibitor of the Bcl-2 family of proteins is AZD5991.

9. The combination, the inhibitor of the Bcl-2 family of proteins, or the aurora kinase inhibitor for use according to any preceding claim, wherein the cancer is breast cancer.

10. The combination, the inhibitor of the Bcl-2 family of proteins, or the aurora kinase inhibitor for use according to any preceding claim, wherein the cancer is non-HER2-enriched breast cancer.

11. The combination, the inhibitor of the Bcl-2 family of proteins, or the aurora kinase inhibitor for use according to any preceding claim, wherein the cancer is determined to be HER2−.

12. The combination, the inhibitor of the Bcl-2 family of proteins, or the aurora kinase inhibitor for use according to any preceding claim, wherein the cancer is basal-like breast cancer.

13. The combination, the inhibitor of the Bcl-2 family of proteins, or the aurora kinase inhibitor for use according to any preceding claim, wherein the cancer is triple negative breast cancer.

14. The combination, the inhibitor of the Bcl-2 family of proteins, or the aurora kinase inhibitor for use according to any of claims 1 to 11, wherein the cancer is luminal B breast cancer.

15. The combination, the inhibitor of the Bcl-2 family of proteins, or the aurora kinase inhibitor for use according to any of claims 1 to 11, wherein the cancer is luminal A breast cancer.

16. The combination, the inhibitor of the Bcl-2 family of proteins, or the aurora kinase inhibitor for use according to any preceding claim, wherein the aurora kinase inhibitor is an aurora A kinase inhibitor.

17. The combination, the inhibitor of the Bcl-2 family of proteins, or the aurora kinase inhibitor for use according to any one of claims 1 to 15, wherein the aurora kinase inhibitor is selected from alisertib, tozasertib, ZM447439, AZD2811, AZD1152, LY3295668, MK-5108, GSK1070916, and MLN8054.

18. The combination, the inhibitor of the Bcl-2 family of proteins, or the aurora kinase inhibitor for use according to claim 17, wherein the aurora kinase inhibitor is selected from alisertib, tozasertib, ZM447439, AZD2811, and AZD1152.

19. The combination, the inhibitor of the Bcl-2 family of proteins, or the aurora kinase inhibitor for use according to claim 17, wherein the aurora kinase inhibitor is selected from alisertib, tozasertib, and ZM447439.

20. The combination, the inhibitor of the Bcl-2 family of proteins, or the aurora kinase inhibitor for use according to claim 17, wherein the aurora kinase inhibitor is alisertib.

21. The combination, the inhibitor of the Bcl-2 family of proteins, or the aurora kinase inhibitor for use according to any preceding claim, wherein the inhibitor of the Bcl-2 family of proteins and the aurora kinase inhibitor are administered separately.
